(19) United States
(12) Patent Application Publication
(10) Pub. No.: US 2021/0121060 A1
(43) Pub. Date: Apr. 29, 2021

(54) VISUAL SYSTEMS ASSESSMENT SCALE AND SYSTEMS AND METHODS FOR CORRESPONDING TREATMENT FOR A PATIENT

(71) Applicant: NORTHWEST EYE CARE, LLC, Clackamas, OR (US)

(72) Inventors: BRUCE WOJCIECHOWSKI, CLACKAMAS, OR (US); JOHN ANTHONY HARTMAN, SHERWOOD, OR (US)

(21) Appl. No.: 17/080,535

(22) Filed: Oct. 26, 2020

Related U.S. Application Data
(60) Provisional application No. 62/925,969, filed on Oct. 25, 2019.

(51) Int. Cl. A61B 3/032; G16H 10/60; G16H 50/30; G16H 50/70; G16H 50/20; G16H 40/67; A61B 3/113; A61B 3/00

(52) U.S. Cl. CPC A61B 3/032; G16H 10/60; G16H 50/30; G16H 50/70; A61B 3/0041; G16H 40/67; A61B 3/113; A61B 3/0025; A61B 3/0091; G16H 50/20

(57) ABSTRACT

A computer-implemented method for performing a vision-related assessment of a patient, the method comprising: receiving information pertaining to each of a plurality of visual assessment-related tests that are performed on the patient, wherein each of the plurality is selected from the group consisting of: Random Dot; Clarity; Near Point; Central Point; Dynamic Acuity; Static Acuity; Northeastern State University College of Optometry (NSUCO) Pursuits; NSUCO Saccades; and Cover; and determining a Status, Symptoms, and Performance (SSP) Score for the patient based at least in part on results of each of the plurality of visual assessment-related tests performed on the patient.

### PRIORITY

This disclosure claims the benefit of U.S. Provisional Application No. 62/925,969, titled “VISUAL SYSTEMS ASSESSMENT SCALE AND SYSTEMS AND METHODS FOR CORRESPONDING TREATMENT FOR A PATIENT” and filed on Oct. 25, 2019, the content of which is incorporated herein by reference in its entirety.

### TECHNICAL FIELD

This disclosure is directed to visual assessment systems and methods, and more particularly to systems and methods for performing visual assessments of a patient and providing corresponding treatment for the patient.

### BACKGROUND

In the United States, approximately 1.5 million people sustain a traumatic brain injury (TBI) every year. Such TBIs can be incurred by falls, vehicle accidents, acts of violence, sport injuries, explosive blasts, and combat injuries, for example. People with TBI suffer from wide-ranging physical and psychological effects that may appear immediately after the injury or at a later time, sometimes much later. Symptoms may include, but are not limited to, loss of consciousness, altered states of consciousness, headaches, nausea and/or vomiting, fatigue and/or drowsiness, changes in sleeping patterns, loss of balance, dizziness, and vision changes, for example. The quality of life of TBI patients may be significantly impacted through such debilitating symptoms.

One of the fundamental processes with which people interact with the world is through the visual system, which is one of the main areas that are affected by TBI. The total loss or even minor impairment of a patient's visual system can cause day-to-day activities, such as cooking, reading, bathing, and buying groceries, to become difficult or, for some people, impossible to complete. Furthermore, when a visual system has been damaged, the damage impacts all systems and the deficits can be devastating as vision is the process of deriving meaning from what is seen. This is a complex system that is learned and developed from childhood and, as such, the ability to rehabilitate such visual impairment is important to help TBI patients recover and return to their lives.

Existing treatments primarily occur in a medical office with the assistance of a visual therapist using traditional vision therapy (VT), which consists mainly of a progressive program of vision exercises, visual stimuli, or procedures that are conducted under the supervision of a therapist. Such treatment may be supplemented with in-home activities to be performed by the patient on his or her own. However, such treatments are limited in efficiency, effectiveness, and overall results.

Conventional platforms are generally focused on the assessment of an assault or other injury (e.g., concussion or other neurological deficits) by way of eye tracking and associated measurements. Such platforms are mostly focused on assessment.

### SUMMARY

Implementations of the disclosed technology are generally directed to systems and methods for measuring human binocular visual perception including hand, eye, body, visual spatial, and visual perception performance in three-dimensional (3D) fields, e.g., in order to create a vision assessment scale that is based on a certain score, e.g., represented by a whole number between one and ten.

Certain implementations of the disclosed technology may include a vision assessment scale (referred to herein as an SSP (Status, Symptoms, and Performance Measures) scale) for assessing a physical deficit or performance variables of human reactions in a volumetric scan, for example.

Certain implementations of the disclosed technology may include a non-invasive medical platform that is suitable for use in connection with telemedicine for diagnostics and treatment, for example.

Certain implementations of the disclosed technology may advantageously lead to improved neurological deficit from multiple vectors.

Certain implementations of the disclosed technology may include the providing of remote access to patient populations, e.g., for assessment and training.

### BRIEF DESCRIPTION OF THE DRAWINGS

FIG. 1 illustrates an example of a visual systems assessment method in accordance with certain implementations of the disclosed technology.

FIG. 2 illustrates an example of a method for enrolling a patient into a visual systems assessment platform in accordance with certain implementations of the disclosed technology.

FIG. 3 illustrates an example of an SSP scoring platform in accordance with certain implementations of the disclosed technology.

FIG. 4 illustrates an example of an SSP scale in accordance with certain implementations of the disclosed technology.

FIG. 5 illustrates an example of a visual systems data collection method in accordance with certain implementations of the disclosed technology.

FIG. 6 illustrates an example of a visual systems assessment platform in accordance with certain implementations of the disclosed technology.

### DETAILED DESCRIPTION

When the human visual system has been damaged (e.g., through accident, injury, etc.), such damage can and often does significantly impact and disrupt the integration of information from any or all of the other human senses. These deficits are typically devastating because vision is the process of deriving meaning from what is seen.

These neurological deficits often lead to a lifetime of problems for the patient. Current treatment methods are disadvantageously based on any or all of the following: manuals, outdated methods, drugs, and expensive solutions.

Because the human visual system has the capacity for change (e.g., neural plasticity) and further because such ability is retained throughout a person's life, the rehabilitation of a person's visual system is entirely possible. Indeed, implementations of the disclosed technology generally include a visual training system that can advantageously repair these deficits and thus improve a person's sensory—and thus complete—human experience.

Certain implementations of the disclosed technology include a solution that is configured to use virtual reality (VR) to leverage the human capacity to repair visual deficits, thus allowing for a proverbial leap frog over current treatment options. Such solution may include a platform customized for the assessment of and treatment of visual and spatial deficits for a given patient, for example.

Certain embodiments may include an assessment and treatment platform that may be designed and built to be advantageously non-invasive, doctor-friendly, patient-friendly, and user-friendly.

Implementations of the disclosed technology generally include a platform that is or can be configured to use a scale of human vision that is referred to herein as a SSP (Status, Symptoms, and Performance Measures) scale. As used herein, Symptoms may refer to a TBI checklist and either or both Performance Measures and Status may refer to bioinformatics information, for example.

Tests may include any or all of the following core vectors: heart rate, galvanic skin response, and oxygen sensor. Alternatively or in addition thereto, tests may include other bioinformatics markers that present themselves in the patient.

In certain embodiments, inputs for the tests may include, but are not limited to, dynamic acuity, pupil response (e.g., alpha and omega color scales), and dynamic visual/color fields (e.g., field assessment).

In certain embodiments, the platform may be configured to perform remote medial assessment and treatment of visual deficits as well as training that can be performed by using both two-dimensional (2D) and three-dimensional (3D) techniques, for example.

The SSP scale typically ranges in value (e.g., in whole numbers) from one to ten. From values of one to five, the scale may be focused on a visual assault from incidents such as TBI, stroke, or other neurological disorders. In cases of neuro-motor impairment as a result of TBI, for example, it is the motor portion of the visual process that is affected and will thus ultimately be involved in recovery. This can also interfere with recovery and rehabilitation when left untreated.

Patients with TBI usually have disruption in magnocellular (e.g., ambient) processing that affects their ability to concentrate on slower moving parvocellular (e.g., focal) processing signals, and they are more likely to be disturbed by the faster moving and now confusing external ambient signals.

TBI patients generally have visual processing difficulties because visual pathways exist throughout the brain and are easily disturbed by trauma. There is usually a disruption in ambient processing as well as an inability for the person to synchronize non-visual subcortical signals with peripheral or central eyesight cortical signals, thereby resulting in non-visual symptoms such as pain, fatigue, sleep disturbances, confusion, dizziness, and aggression.

Implementations of the disclosed technology generally include the use of bio-mechanical data points, gaze vectors, heart rate, pupil movements, midline, and neurotropic and psychotropic effects of prescribed and un-prescribed substances and other factors to measure against.

Certain implementations are directed to a training platform that includes the use of “Vision Rooms” for using visual tracking technologies as well as corresponding training methodologies. In certain embodiments, the training may be geared toward repairing spatial/visual mismatches.

The following factors may be plotted on an SSP scale: Status, Symptoms, and Performance Measures. This provides three data points to measure against and also accounts for variation in the natural human visual system.

Certain implementations may include the use of a three color system to indicate at a glance where the current status of the person in relation to norms. For example, green may be used to indicate that all three factors are in the normal zone, yellow may be used to indicate that at least one of the factors is outside of the norm, and red may be used to indicate that all three factors are outside of the norms.

While certain colors are discussed herein, it will be appreciated that the colors used by the system are not necessarily limited to green, yellow, and red, and further that virtually any set of colors, patterns, signals, etc. may be used in alternative implementations of the disclosed technology.

Normal vision generally falls at the five to six mark on the one-to-ten SSP scale. When moving into the six-to-ten mark, the SSP scale becomes more about measuring performance. Implementations may include tools for ensuring that the measured results are accurate.

The disclosed “Vision Rooms” may be designed to level up in accordance with the patient's progress. For example, the patient may be assessed after being at a certain level for a certain period of time and, based on where the user then falls on the SSP scale, the same level may be repeated, or the patient may move onto either the next higher or lower “Vision Room” level.

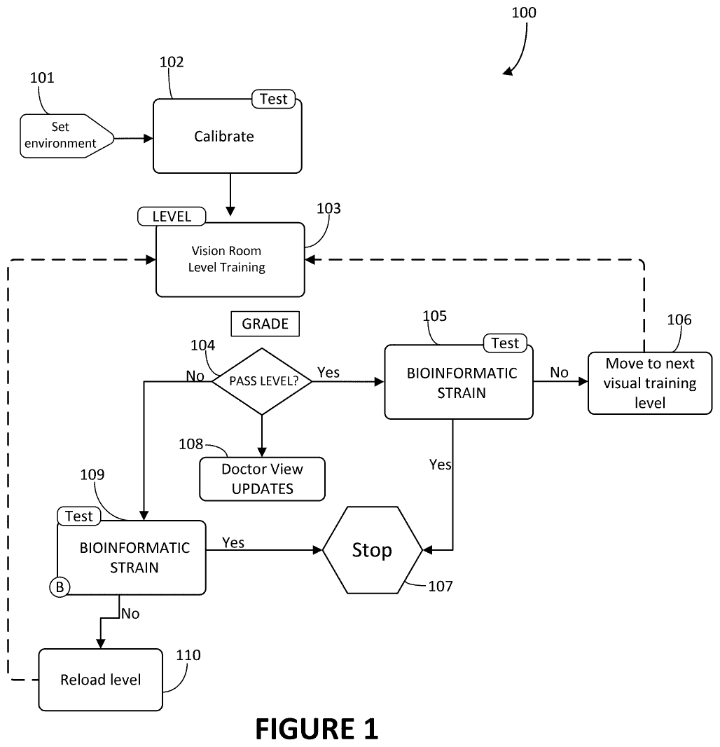

FIG. 1 illustrates an example of a visual systems assessment method 100 in accordance with certain implementations of the disclosed technology. In the example, the method begins by setting the environment, as indicated at 101. A calibration (e.g., calibration of a VR unit) may then be performed, as indicated at 102.

A “Vision Room” level training may then be performed, as indicated at 103, and a grade (e.g., pass/fail) may be determined for the training. A determination may be made as to whether the grade is a passing grade, as indicated at 104: if the grade for the “Vision Room” training is a non-passing grade, the level may be maintained and a subsequent assessment may be performed, as indicated at 109; if, however, the grade for the “Vision Room” training is a passing grade, a bioinformatic strain (e.g., eye strain) testing may be performed, as indicated at 105.

If the grade for the bioinformatic strain testing is a passing grade, the method may move to the next visual training level for the “Vision Room” training, as indicated at 106; if the grade for the bioinformatics strain testing is a non-passing grade, the method may issue a “stop” command and inform the patient that it is time for a break, as indicated at 107.

Regardless of the grade determined for each “Vision Room” assessment, an update may be generated and optionally presented or otherwise delivered to a doctor or other authorized user and/or presented/saved in a report.

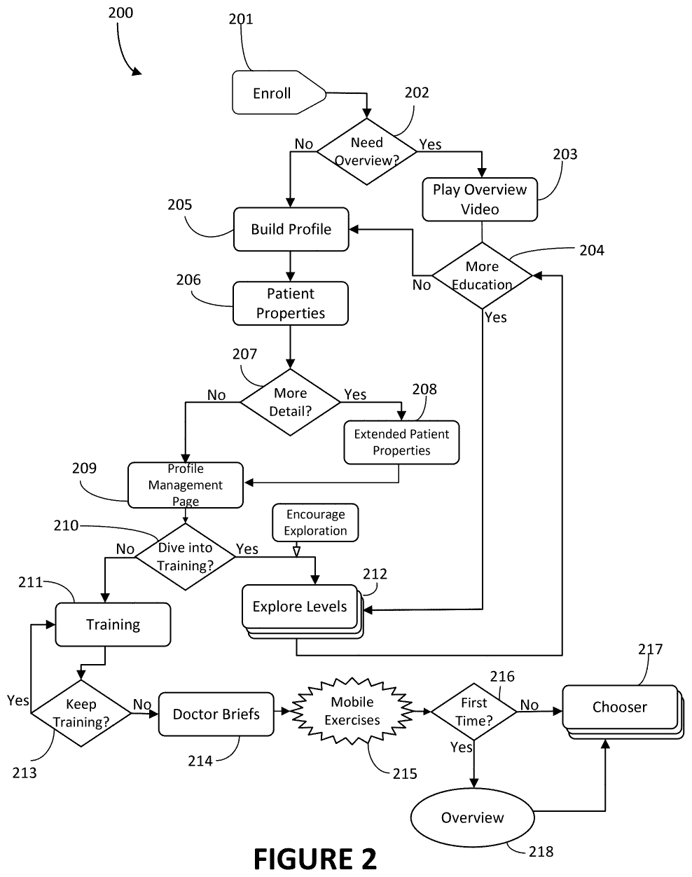

FIG. 2 illustrates an example of a method 200 for enrolling a patient into a visual systems assessment platform in accordance with certain implementations of the disclosed technology. In an initial operation, a patient may begin the enrollment process, as indicated at 201. A determination is made as to whether the patient needs an overview, as indicated at 202: if the patient needs an overview, the system may play an overview video for the patient, as indicated at 203 and a determination may be made as to whether more education is needed, as indicated at 204; if more education is not needed, the system may direct the patient to build a profile, as indicated at 205; otherwise, the method may proceed to explore levels, as indicated at 212.

The system may be configured to obtain a certain amount of information from the patient including extended patient properties, as indicated at 206. A determination may be made as to whether more details are needed, as indicated at 207: if yes, extended patient properties may be obtained, as indicated at 208; if not, the patient may be directed to a profile management page, as indicated at 209.

A determination may be made as to whether training may begin, as indicated at 210: if so, the method proceeds to 212; if not, the method proceeds to training at 211. Once the profile has been created, the system may begin a training portion, as indicated at 211 and optionally provide a doctor and/or other authorized user with briefs, as indicated at 214. The method may proceed to mobile exercises, as indicated at 215 and a determination may be made as to whether this is the user's first time, as indicated at 216: if so, the method may proceed to an overview, as indicated at 218; if not, the method may proceed to a chooser at 217.

Certain implementations of the disclosed technology serve to simplify 3D tools and use a game engine to assess a patient's visual and physical measurements. A scale based on Symptom, Status, and Performance (SSP Scale) results in an entirely new paradigm by digitizing and automating advanced neuro-visual and cognitive testing using eye-tracking hardware and custom software. Implementations may also include a digital portal that connects any device into a remote training experience.

Certain implementations may include a digitized test suite in which standard analog ophthalmological tests are digitized. The modalities for digitization may include: camera only (e.g., a web-based machine learning (ML) approach to harvesting data; via application (e.g., mobile applications taking advantage of more complex sensors, AI, and data capture); and virtual reality (VR). There are varying degrees of precision for these modalities to be accounted for, which may be calculated in a scoring algorithm. Any or all of the following raw eye data may be collected: frame time, combined origin XYZ, left origin XYZ, right origin XYZ, combined direction XYZ, right direction XYZ, left direction XYZ, right openness, left openness, right pupil diameter, left pupil diameter, right pupil XY, left pupil XY, and actions associated with gaze triggers (i.e., input triggers).

In certain implementations, a suite of tests are performed on a patient using eye-tracking HUD, for example. Such test may include—but are not limited to—the following: Random dot, Clarity, Near Point, Central Point, Dynamic Acuity, Static Acuity, NSUCO Pursuits, NSUCO Saccades, and Cover.

Certain implementations include a set of software tools and frameworks to extend standard Content and Learning Management software that may include storytelling to improve the participant's retention on the portal, for example, using the same backend that some studios use to manage story and engagement. This new modality may advantageously provide a visual performance measurement space akin similar to physical athletic combines.

The Ganzfeld method of testing is unique (e.g., an unstructured, uniform stimulus field) and significant in enabling the ability to test visual forms objectively of visual input processing. Prior visual testing brings in multiple biases to the testing platform and there is no uniformity when it comes to testing ocular alignment, heterophorias, vergence eye movements, visual acuity, pursuit and saccadic eye movements, stereopsis, depth of field, and dynamic visual acuity. The many biases involved in these prior tests include, but are not limited to, the following: lighting, size of the testing room, proximity of the examiner, centralperipheral disturbances (i.e., visual clutter), objects in the room, examiner skill, patient reliability, etc.

The uniqueness of being in a Ganzfeld setting allows implementations to create uniform visual metrics (e.g., posture, stamina, speed, amplitude, flexibility, and fixation ability) of the human system in its highest and most pure form of visual function. Whereas prior tests of visual performance were only tested in the x and y axis and have no uniformity and less objectivity, implementations of the disclosed technology allow testing in the z axis as well. This advantageously provides the unique ability to test pure visual skills and visual input processing.

Implementations may use bio-mechanical data points, gaze vectors, heart rate, pupil movements, midline, and other factors to measure users.

Certain implementations may include a mobile testing suite that is able to take advantage of gyroscope, LIDAR and other camera and in-device technologies, and a web camera via a digital portal, for example. Machine learning (ML) and other techniques may be used in certain implementations.

Information may be collected via self-reporting questionnaires based on a categories of collection. Questionnaire examples include: BVISS; ACES and other TBI and neurological assessment questions; Aging in Place Assessment; and Esports Pro Athlete Assessment. Profile information of the user may also be collected. Comparatives may be pulled from the profile according to demographics. Self-reporting compared to the test results may be considered to adjust for blatant mis-reporting.

A user's status score may be captured by data collected across a spectrum of inputs including any or all of the following: automation assessment; guided assessment with technician; and a combination of both. Physical measurement from the tests collected may be used to score each test taken. A benchmark may be created or a previous score may be compared. The fidelity or accuracy of the test performed may be measured.

Implementations may include measuring how a participant fairs against others in hand-eye, body-brain, and visual spatial reactions in 3D space, for example. Results may be compared against the populous based on calculated norms. A comparison may be performed across the collected demographic spectrum, and an overall quality of harvest metric evaluation may be performed to achieve a performance metric.

In certain implementations, a user may be assessed after a level and, based on where they fall on the SSP scale, the level may be repeated or the user may be moved onto the next level. With the SSP scale, particular problematic areas may be isolated and specific therapies for those shortcomings in human visual plasticity may be targeted. Visual shortcomings may be identified, quantified, and rectified. The scale and treatment may advantageously identify particular areas that are missed.

Visual strategies may be enhanced, retrained, or developed for each individual. Other therapy modalities, such as speech and language visual-spatial visual-vestibular pathways, may be integrated. Assistive technology to enhance visual shortcomings may be developed.

The scale may be used to help players focus their skills, help teams evaluate player ability, and improve fan understanding of overall player performance. Certain implementations may include a Gamer Vision Rank (GVR) and a tele-training portal for athletes, coaches, and teams to manage the assessment and training of visual performance, e.g., how they focus, what might be causing them fatigue, and what therapy will help them gain the performance edge, for example.

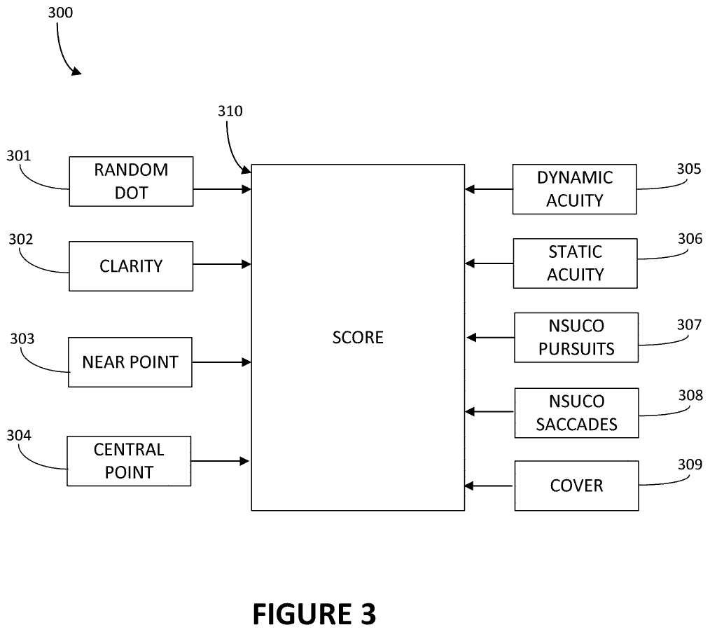

FIG. 3 illustrates an example of an SSP scoring platform 300 in accordance with certain implementations of the disclosed technology. In the example, any or all of nine different inputs may be taken into account in determining an SSP Score 310 for a patient: Random Dot 301, Clarity 302, Near Point 303, Central Point 304, Dynamic Acuity 305, Static Acuity 306, NSUCO Pursuits 307, NSUCO SACCADES 308, and Cover 309.

With regard to Individual Clarity 302, there are typically five letters in front of the patient. When the patient converges their eyes on a letter, it is ‘extinguished.’ When all five letters are extinguished, 5 more letters appear in a smaller size. The total number of letters extinguished and the smallest size reached may be determined.

With regard to Near Point 303, a sphere is typically moved close to the patient's face. The patient then focuses their eyes on the sphere, and when the sphere ‘becomes two’ the patient presses the trigger. The sphere then moves away from the user's face and, when the sphere ‘becomes one’ again, they press the trigger. This may be repeated multiple times. Each time the user presses the trigger, the distance of the sphere from the user's face may be measure. The average of the ‘doubling’ and ‘return’ values may be reported.

Convergence generally refers to neuromuscularly executed—but cortically-guided—act that keeps both of the patient's eyes trained on a target as it approaches on the z-axis; that is, the higher-level areas of the patient's brain respond to the stimulus to converge (e.g., stimulus=temporary/momentary double vision), and send the signal to the extraocular muscles [that control eye movements] with a positive feedback loop to stimulate each eye to look directly at the target, thereby providing single vision. Convergence is essential for the patient to avoid double vision (i.e., diplopia) and also to provide the patient's brain with real-time information about target location.

Poor Near Point convergence generally results in ill-sustained Near Point activities (e.g., looking at screens), eye pain and strain, headache, avoidance of tasks, sleepiness when reading or watching screens, double vision, and poor depth perception. Poor Near Point convergence can also be associated with poor Far Point convergence, which can typically negatively affects depth perception, body coordination, visual acuity, and can also cause the symptoms noted above.

With regard to Central Point 304, the patient typically looks ahead at a sphere in the center of their vision. When their eyes converge on that sphere, another sphere comes in from their peripheral vision. When the user pulls the trigger, the distance from that sphere to the center may be measured. The average of these distances may be determined.

Peripheral awareness, as opposed to central vision, is generally coordinated by a different type of cell in a patient's retina that has connections to other sensory systems that are in communication with the patient's body as to where the person is in space; that is, peripheral vision is a key component of the “righting” system that allows a patient to stay upright and balanced. Specialized cells in the peripheral retina form a neurological pathway called the magnocellular pathway, which diverges from the central vision and integrates with areas of the brain that process motion, balance, spatial recognition, and localization.

Peripheral vision is also important for planning saccadic eye movements; without proper peripheral awareness, such quick, ballistic eye movements [that cannot be adjusted mid-movement] do not have an accurate target and, thus, many small corrective eye movements are often needed to locate a target without such accurately-planned motion. With strong peripheral vision awareness, such quick eye movements are more accurate.

Dynamic Acuity 305 and Static Acuity 306 are similar to Clarity 302 except symbols are used instead of letters. A symbol is typically presented in front of the patient, and their goal is to converge their eyes on the matching symbol. In Static Acuity these are fixed, and in Dynamic Acuity they move around. The percentage of correct answers and smallest size reached may be determined.

A visual acuity test is generally a measurement of image resolution of each eye which is also referred to as the “20/20 ability” of a patient's eyes. The test is typically affected by optical blur, edge interpretation ability, and focus accuracy. Disruptions to visual acuity generally include uncompensated or inaccurately compensated refractive error (e.g., nearsightedness and astigmatism), opacity in the media of the eyes (e.g., corneal disease), retinal irregularities (e.g., both congenital and acquired), and functional visual dysfunction (e.g., poorly-developed connection between the patient's eyes and brain.

Testing visual acuity is important because it could be a limiting factor in a patient's visual function if the image from one or both eyes is blurred enough to compromise accurate localization, anticipation, or interpretation of a target (either static or in motion). Visual acuity is important in image interpretation, especially with regard to identification. Dynamic acuity is usually important for elite athletes, pilots, and other high-risk situations. In active situations, dynamic acuity is generally more important because static acuity (i.e., static subject and static target) are less relevant. The difference between static and dynamic acuity may be tracked over time and/or pre- or post-intervention.

With regard to NSUCO Pursuits 307, a sphere is generally moved in circles and lines ahead of the patient. The percentage of time the user's eyes are converged on the sphere may be tracked. A second score value for this test may be calculated as follows: using the raw eye sensor motion data, a time series of (x,y) coordinates of the eyes may be obtained; a smoothed copy of the time series may be generated; and the difference of the raw time series of the smoothed time series may be determined.

With regard to NSUCO Saccades 308, there are typically two spheres in front of the patient, who is told to look from one to the other. Overshoot and undershoot may be measured, where undershoot generally means that the eye stops part of the way then moves the rest of the way, and overshoot generally means the eye stops after the target and moves backwards. To calculate these, a determination is generally made as to whether the patient's eye stops multiple times as they move from one sphere to another: if their eyes go straight to the center of the target and stay there, there is no over/undershoot; but if their eyes stop twice or more, where the eye ‘rests’ is taken to be the ‘center’ of the target. The overshoot and undershoot are generally the most extreme differences between where the eye rests and the other point(s) the eye stopped.

Saccades are generally rapid, ballistic movements of a patient's eyes that abruptly change the point of fixation. They may range in amplitude from small movements made while reading, for example, to larger movements made while gazing around a room, for example. Saccades may be elicited voluntarily but typically occur reflexively when the patient's eyes are open, even when fixated on a target. Saccadic eye movements make up the majority of all eye movements the patient elicits in his or her daily life, regardless of the task. Accurate, precise, and well-timed saccades are more important in elite sports and other high-demand visual tasks.

With regard to Cover 309, the patient typically looks at a sphere right in front of their face. Each eye's display is alternately turned on and off. This is ‘covering’ each eye one at a time, and the patient's eyes will begin to move each time the display is switched to the other eye. The degree of motion that occurs because of this may be measured. This may be scored similarly to NSUCO Pursuits but without the ‘percentage of time looking at the sphere’ metric.

Simultaneous Tracking is similar to Central Point except that the sphere is typically moving in a circle. The sphere that moves in from the sides starts fast, and slows at it reaches its destination. The patient must converge their eyes on that sphere instead of pulling the trigger. The distance between the peripheral sphere and its destination may be measured and the average of these distances may be determined.

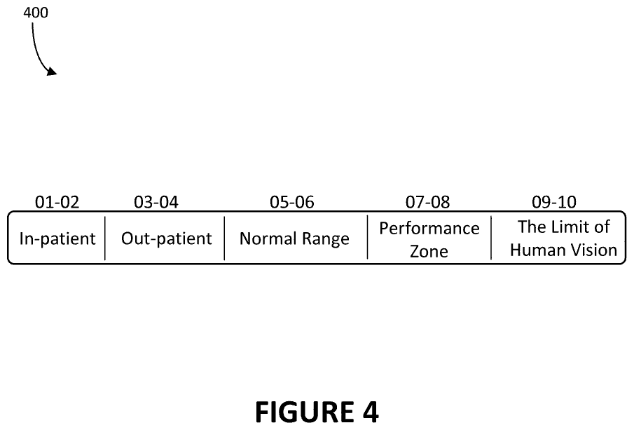

FIG. 4 illustrates an example of an SSP scale 400 that ranges from 01-10 in accordance with certain implementations of the disclosed technology. In the example 400, there are five zones: a first zone (e.g., scores between 01-02) that generally represents in-patient, a second zone (e.g., scores between 03-04) that generally represents out-patient, a third zone (e.g., scores between 05-06) that generally represents a normal ranges, a fourth zone (e.g., scores between 07-08) that generally represents a performance zone, and a fifth zone (e.g., scores between 09-10) that generally represents the limit of human vision.

Use of the SSP scale advantageously provides three data points to measure against and account for variation in the natural human visual system. Certain implementations may include a multi-color system (e.g., three-color system) to indicate at a glance where the patient falls in relation to the norms. For example, green may be used to indicate that all three factors are in the normal zone, yellow may be used to indicate that at least one of the factors is outside of the norm, and red may be used to indicate that all three factors are outside of the norms.

From one to five, the SSP scale generally presents as visual assault from things like TBI, Stroke, or other neurological disorders. In cases of neuro-motor impairment resulting from traumatic brain injury, it is the motor portion of the visual process that is affected and will ultimately be involved in recovery. It can also interfere with recovery and rehabilitation when left untreated. Normal vision generally falls at the five to six mark on the SSP scale. In the six to ten mark, the scale is generally about measuring performance. These metrics may be obtained by VR HUD eye tracking tests, which typically collect the most precise data.

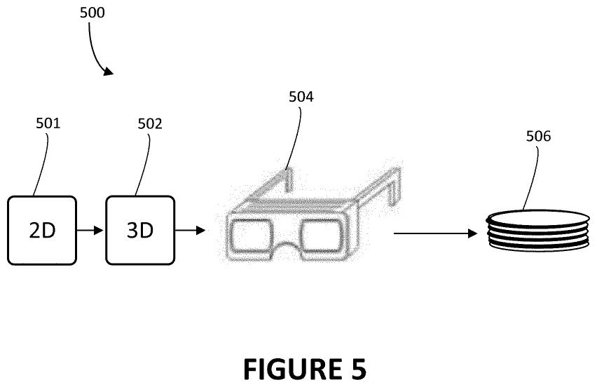

FIG. 5 illustrates an example of a visual systems data collection method 500 in accordance with certain implementations of the disclosed technology. In the example 500, patient-specific information and/or data may be harvested from a two-dimensional (2D) source such as an iPad or a laptop, as indicated by 501. Alternatively or in addition thereto, patient-specific information and/or data may be harvested from a three-dimensional (3D) source such as a virtual reality (VR) eye menu, as indicated by 502.

At 504, a VR device such as a VR eye-tracking HUD or other suitable device may be used to perform as assessment such as any or all of the following: 20/20; divergence-convergence; tracking and teaming; fixation; pupil drift; depth of field; retinoblastoma screening; and cognitive recall. Any or all of the harvested data may be stored in a storage device 506, which may be local or remote with regard to the system.

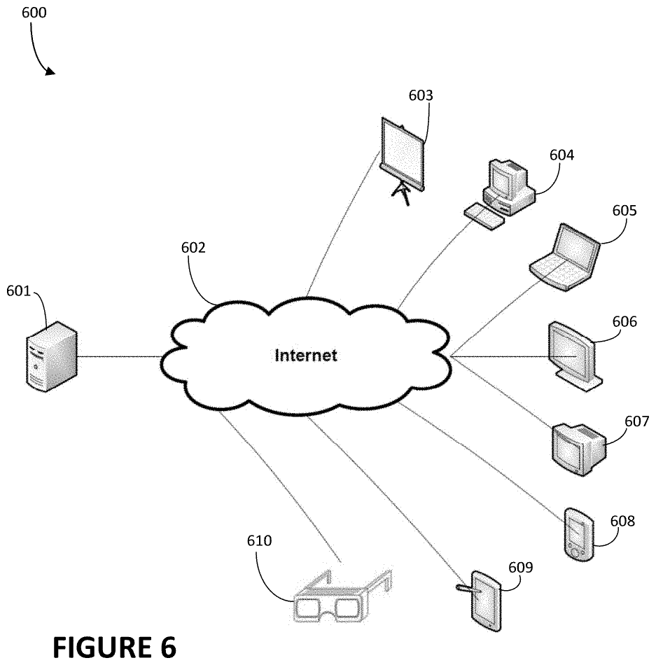

FIG. 6 illustrates an example of a visual systems assessment platform 600 in accordance with certain implementations of the disclosed technology. It will be understood that the components of the illustrated system are merely exemplary in nature, and that systems and methods may be employed in any suitable environment.

A number of different devices may be available to allow a patient to use or otherwise access implementations of the disclosed visual assessment system. Such devices may include a desktop computer 604, a laptop computer 605, a cell phone or smart phone 608, other portable devices 609, a television 606, a tablet device 609, virtual reality goggles 610, a projector screen 603, and other types of computing devices 601 and 607, for example. It should be understood that any or all of these devices may be used in isolation or in any combination with any or all of the other devices.

Aspects of the disclosure may operate on particularly created hardware, firmware, digital signal processors, or on a specially programmed computer including a processor operating according to programmed instructions. The terms controller or processor as used herein are intended to include microprocessors, microcomputers, Application Specific Integrated Circuits (ASICs), and dedicated hardware controllers.

One or more aspects of the disclosure may be embodied in computer-usable data and computer-executable instructions, such as in one or more program modules, executed by one or more computers (including monitoring modules), or other devices. Generally, program modules include routines, programs, objects, components, data structures, etc. that perform particular tasks or implement particular abstract data types when executed by a processor in a computer or other device. The computer executable instructions may be stored on a computer readable storage medium such as a hard disk, optical disk, removable storage media, solid state memory, Random Access Memory (RAM), etc. As will be appreciated by one of skill in the art, the functionality of the program modules may be combined or distributed as desired in various aspects. In addition, the functionality may be embodied in whole or in part in firmware or hardware equivalents such as integrated circuits, FPGAs, and the like.

Particular data structures may be used to more effectively implement one or more aspects of the disclosure, and such data structures are contemplated within the scope of computer executable instructions and computer-usable data described herein.

The disclosed aspects may be implemented, in some cases, in hardware, firmware, software, or any combination thereof. The disclosed aspects may also be implemented as instructions carried by or stored on one or more or computer-readable storage media, which may be read and executed by one or more processors. Such instructions may be referred to as a computer program product. Computer-readable media, as discussed herein, means any media that can be accessed by a computing device. By way of example, and not limitation, computer-readable media may comprise computer storage media and communication media.

Computer storage media means any medium that can be used to store computer-readable information. By way of example, and not limitation, computer storage media may include RAM, ROM, Electrically Erasable Programmable Read-Only Memory (EEPROM), flash memory or other memory technology, Compact Disc Read Only Memory (CD-ROM), Digital Video Disc (DVD), or other optical disk storage, magnetic cassettes, magnetic tape, magnetic disk storage or other magnetic storage devices, and any other volatile or nonvolatile, removable or non-removable media implemented in any technology. Computer storage media excludes signals per se and transitory forms of signal transmission.

Communication media means any media that can be used for the communication of computer-readable information. By way of example, and not limitation, communication media may include coaxial cables, fiber-optic cables, air, or any other media suitable for the communication of electrical, optical, Radio Frequency (RF), infrared, acoustic or other types of signals.

### EXAMPLES

Illustrative examples of the technologies disclosed herein are provided below. An embodiment of the technologies may include any one or more, and any combination of, the examples described below.

Example 1 is a computer-implemented method for performing a vision-related assessment of a patient, and includes: receiving information pertaining to each of a plurality of visual assessment-related tests that are performed on the patient, wherein each of the plurality is selected from the group consisting of: Random Dot; Clarity; Near Point; Central Point; Dynamic Acuity; Static Acuity; Northeastern State University College of Optometry (NSUCO) Pursuits; NSUCO Saccades; and Cover; and determining a Status, Symptoms, and Performance (SSP) Score for the patient based at least in part on results of each of the plurality of visual assessment-related tests performed on the patient.

Example 2 is the computer-implemented method of Example 1, wherein performing the Clarity test on the patient includes visually presenting to the patient a plurality of characters that are ‘extinguished’ when the patient converges their eyes on a letter, then visually presenting to the patient at least one successive plurality of characters in a progressively smaller size until a smallest size of the characters reached is determined.

Example 3 is the computer-implemented method of any of Examples 1-2, wherein performing the Near Point test on the patient includes physically moving a sphere close to the patient's face such that the patient focuses their eyes on the sphere until the sphere appears to split into two spheres, then moving the sphere away from the user's face until the sphere appears to become one sphere again.

Example 4 is the computer-implemented method of any of Examples 1-3, wherein performing the Central Point test on the patient includes having the patient look ahead at a sphere in the center of their vision such that another sphere comes in from the patient's peripheral vision when their eyes converge on that sphere.

Example 5 is the computer-implemented method of any of Examples 1-4, wherein performing the Dynamic Acuity test on the patient includes visually presenting to and in front of the patient a moving symbol until the patient converges their eyes on a matching symbol.

Example 6 is the computer-implemented method of any of Examples 1-5, wherein performing the Static Acuity test on the patient includes visually presenting to and in front of the patient a positionally fixed symbol until the patient converges their eyes on a matching symbol.

Example 7 is the computer-implemented method of any of Examples 1-6, wherein performing the NSUCO Pursuits test on the patient includes moving a sphere in circles and lines ahead of the patient and tracking a percentage of time the user's eyes are converged on the sphere.

Example 8 is the computer-implemented method of any of Examples 1-7, wherein performing the NSUCO Saccades test on the patient includes presenting two spheres to and in front of the patient and instructing the patient to look from one sphere to the other, and measuring overshoot and undershoot.

Example 9 is the computer-implemented method of any of Examples 1-8, further comprising storing the information pertaining to each of the plurality of visual assessment-related tests that are performed on the patient.

Example 10 is the computer-implemented method of any of Examples 1-9, further comprising locally and/or remotely storing the SSP Score for the patient.

Example 11 is the computer-implemented method of any of Examples 1-10, wherein the SSP Score corresponds to an SSP Scale that includes a plurality of sub-portions.

Example 12 is the computer-implemented method of Example 11, wherein the SSP Scale has a range between 1-10.

Example 13 is the computer-implemented method of any of Examples 11-12, wherein the plurality of sub-portions includes a first sub-portion corresponding to in-patient, a second sub-portion corresponding to out-patient, a third sub-portion corresponding to a normal range, a fourth sub-portion corresponding to a performance zone, and a fifth sub-portion corresponding to a limit of human vision.

Example 14 is the computer-implemented method of any of Examples 11-13, wherein the plurality of sub-portions includes a first sub-portion corresponding to SSP Scores of 1 and 2, a second sub-portion corresponding to SSP Scores of 3 and 4, a third sub-portion corresponding to SSP Scores of 5 and 6, a fourth sub-portion corresponding to SSP Scores of 7 and 8, and a fifth sub-portion corresponding to SSP Scores of 9 and 10.

Example 15 is the computer-implemented method of any of Examples 11-14, wherein the SSP Scale is coded by a plurality of different colors.

Example 16 is the computer-implemented method of Example 15, wherein at least a first sub-portion is identified by red, at least a second sub-portion is identified by yellow, and at least a third sub-portion is identified by green.

Example 17 is the computer-implemented method of any of Examples 1-16, further comprising adjusting at least one treatment for the patient based at least in part on the SSP Score for the patient.

Example 18 is the computer-implemented method of Example 17, further comprising: receiving information pertaining to each of a subsequent plurality of visual assessment-related tests that are performed on the patient; and determining an updated SSP Score for the patient based at least in part on results of each of the subsequent plurality of visual assessment-related tests performed on the patient.

Example 19 is the computer-implemented method of Example 18, further comprising adjusting at least one treatment for the patient based at least in part on the updated SSP Score for the patient.

Example 20 is a system for performing a vision-related assessment of a patient, and includes: a virtual reality (VR) device configured to perform a plurality of visual assessment-related tests on the patient; and one or more processors configured to: receive information pertaining to each of the plurality of visual assessment-related tests that are performed on the patient; and determine a Status, Symptoms, and Performance (SSP) Score for the patient based at least in part on results of each of the plurality of visual assessment-related tests performed on the patient.

The previously described versions of the disclosed subject matter have many advantages that were either described or would be apparent to a person of ordinary skill. Even so, these advantages or features are not required in all versions of the disclosed apparatus, systems, or methods.

Additionally, this written description makes reference to particular features. It is to be understood that the disclosure in this specification includes all possible combinations of those particular features. Where a particular feature is disclosed in the context of a particular aspect or example, that feature can also be used, to the extent possible, in the context of other aspects and examples.

Also, when reference is made in this application to a method having two or more defined steps or operations, the defined steps or operations can be carried out in any order or simultaneously, unless the context excludes those possibilities.

Although specific examples of the invention have been illustrated and described for purposes of illustration, it will be understood that various modifications may be made without departing from the spirit and scope of the invention. Accordingly, the invention should not be limited except as by the appended claims.

We claim:

1. A computer-implemented method for performing a vision-related assessment of a patient, the method comprising:
receiving information pertaining to each of a plurality of visual assessment-related tests that are performed on the patient, wherein each of the plurality is selected from the group consisting of: Random Dot; Clarity; Near Point; Central Point; Dynamic Acuity; Static Acuity; Northeastern State University College of Optometry (NSUCO) Pursuits; NSUCO Saccades; and Cover; and
determining a Status, Symptoms, and Performance (SSP) Score for the patient based at least in part on results of each of the plurality of visual assessment-related tests performed on the patient.

2. The computer-implemented method of claim 1, wherein performing the Clarity test on the patient includes visually presenting to the patient a plurality of characters that are ‘extinguished’ when the patient converges their eyes on a letter, then visually presenting to the patient at least one successive plurality of characters in a progressively smaller size until a smallest size of the characters reached is determined.

3. The computer-implemented method of claim 1, wherein performing the Near Point test on the patient includes physically moving a sphere close to the patient's face such that the patient focuses their eyes on the sphere until the sphere appears to split into two spheres, then moving the sphere away from the user's face until the sphere appears to become one sphere again.

4. The computer-implemented method of claim 1, wherein performing the Central Point test on the patient includes having the patient look ahead at a sphere in the center of their vision such that another sphere comes in from the patient's peripheral vision when their eyes converge on that sphere.

5. The computer-implemented method of claim 1, wherein performing the Dynamic Acuity test on the patient includes visually presenting to and in front of the patient a moving symbol until the patient converges their eyes on a matching symbol.

6. The computer-implemented method of claim 1, wherein performing the Static Acuity test on the patient includes visually presenting to and in front of the patient a positionally fixed symbol until the patient converges their eyes on a matching symbol.

7. The computer-implemented method of claim 1, wherein performing the NSUCO Pursuits test on the patient includes moving a sphere in circles and lines ahead of the patient and tracking a percentage of time the user's eyes are converged on the sphere.

8. The computer-implemented method of claim 1, wherein performing the NSUCO Saccades test on the patient includes presenting two spheres to and in front of the patient and instructing the patient to look from one sphere to the other, and measuring overshoot and undershoot.

9. The computer-implemented method of claim 1, further comprising storing the information pertaining to each of the plurality of visual assessment-related tests that are performed on the patient.

10. The computer-implemented method of claim 1, further comprising locally and/or remotely storing the SSP Score for the patient.

11. The computer-implemented method of claim 1, wherein the SSP Score corresponds to an SSP Scale that includes a plurality of sub-portions.

12. The computer-implemented method of claim 11, wherein the SSP Scale has a range between 1-10.

13. The computer-implemented method of claim 11, wherein the plurality of sub-portions includes a first sub-portion corresponding to in-patient, a second sub-portion corresponding to out-patient, a third sub-portion corresponding to a normal range, a fourth sub-portion corresponding to a performance zone, and a fifth sub-portion corresponding to a limit of human vision.

14. The computer-implemented method of claim 11, wherein the plurality of sub-portions includes a first sub-portion corresponding to SSP Scores of 1 and 2, a second sub-portion corresponding to SSP Scores of 3 and 4, a third sub-portion corresponding to SSP Scores of 5 and 6, a fourth sub-portion corresponding to SSP Scores of 7 and 8, and a fifth sub-portion corresponding to SSP Scores of 9 and 10.

15. The computer-implemented method of claim 11, wherein the SSP Scale is coded by a plurality of different colors.

16. The computer-implemented method of claim 15, wherein at least a first sub-portion is identified by red, at least a second sub-portion is identified by yellow, and at least a third sub-portion is identified by green.

17. The computer-implemented method of claim 1, further comprising adjusting at least one treatment for the patient based at least in part on the SSP Score for the patient.

18. The computer-implemented method of claim 17, further comprising:
receiving information pertaining to each of a subsequent plurality of visual assessment-related tests that are performed on the patient; and
determining an updated SSP Score for the patient based at least in part on results of each of the subsequent plurality of visual assessment-related tests performed on the patient.

19. The computer-implemented method of claim 18, further comprising adjusting at least one treatment for the patient based at least in part on the updated SSP Score for the patient.

20. A system for performing a vision-related assessment of a patient, the system comprising:
a virtual reality (VR) device configured to perform a plurality of visual assessment-related tests on the patient; and
one or more processors configured to:
receive information pertaining to each of the plurality of visual assessment-related tests that are performed on the patient; and
determine a Status, Symptoms, and Performance (SSP) Score for the patient based at least in part on results of each of the plurality of visual assessment-related tests performed on the patient.
